(19) United States
(12) Patent Application Publication
(10) Pub. No.: US 2014/0357529 A1
(43) Pub. Date: Dec. 4, 2014

(54) Microarray and Method for Forming the Same

(71) Applicant: National University of Singapore, Singapore (SG)

(72) Inventors: Wee Kiong Choi, Singapore (SG); Heng Phon Too, Singapore (SG); Raj Rajagopalan, Singapore (SG); Lihan Zhou, Singapore (SG); Mohammed Khalid Bin Dawood, Singapore (SG); Han Zheng, Singapore (SG); He Cheng, Singapore (SG)

(73) Assignee: National University of Singapore, Singapore (SG)

(21) Appl. No.: 14/365,881

(22) PCT Filed: Dec. 19, 2012
(86) PCT No.: PCT/SG2012/000480

§ 371 Date: Jun. 16, 2014

Related U.S. Application Data
(60) Provisional application No. 61/577,171, filed on Dec. 19, 2011.

(51) Int. Cl. C12Q 1/68

(52) U.S. Cl. CPC C12Q 1/6837
USPC 506/16; 506/32

(57) ABSTRACT

There is provided a microarray comprising a plurality of active agents immobilized onto an array of porous nanostructures, wherein each nanostructure has a network of pores that extends throughout the thickness of said nano structure.

### TECHNICAL FIELD

The present invention generally relates to a microarray. The present invention also relates to a method for forming the microarray. The present invention also relates to a system incorporating the microarray and a microfluidic device.

### BACKGROUND

Microarrays are analytical or functional devices that are often used in assaying biological or chemical molecules. These devices are usually made up of monolithic, flat surface substrates that bear hundreds or even thousands of multiple probe sites. Each of these probe sites usually comprise a reagent which is able to molecularly recognize or react with a molecule, which is sometimes referred to as a target. The interaction of the probe to the target produces a signal that can be detected through a number of ways such as by fluorescence, radioactivity or chemi-luminescence, etc.

Microarrays with flat surface substrates suffer from a limitation in that the detection sensitivity is often low. This is because the performance of such microarrays may be compromised due to the undesirable reagent surface interactions as a result of the random nature of the attachment of active agents to the substrate, which may cause some of the immobilized probes or targets to lose their binding affinity/activity (which can be viewed as binding between the probes and the substrate or between the probes and their targets). The poor binding of the probes or targets to the substrate may be due to several factors, such as direct chemical modification of the binding sites, steric hindrance by the surface or adjacent immobilized probes, or the denaturation of the probes themselves. Some examples of these probes may include proteins, DNA and antibodies and the combinations of these molecules etc. In addition, the molecule loading surface area of the substrates is limited, so that only a limited number of molecules loaded on the surface can participate in desired molecule recognition/reaction. Consequently, a flat surface substrate hinders high sensitivity due to the insufficient binding of the probes to the substrates

To mitigate the above issues, a variety of methods in fabricating microarrays on substrates have been proposed. One approach is to alter the surface roughness or geometrical morphology of the substrate, thereby increasing the surface area of the substrate to increase the binding capacity and density of the probes per site. Several complex fabrication methods such as combining thermal deposition, electron beam lithography and reactive ion etching have been explored to increase the surface area for greater binding capacity and density. Although the various array designs and fabrication methods mentioned increase the immobilized probe concentration, and hence the number of sites that are available for target-probe recognition/reaction, they do not address the problem of inaccessibility of the targets arising from the diffusion limitation of biomolecules. Poor accessibility of the targets to probes can result in poor sensitivity and deficient signals. In addition, the above mentioned efforts cannot overcome the unfavorable probe-substrate interactions mentioned before.

Holistically, the several factors mentioned above including, but not limited to, biocompatibility, molecule interaction with substrate, molecule diffusion and geometrical morphology of substrate used, can affect the detection efficiency and hence signal-to-noise ratio of the microarray analysis. In addition, the tailoring of fabrication method depends greatly on the application of the microarray itself.

Accordingly, there is a need to provide a low cost and scalable micro-fabricated device that has wide applications for microarray analysis.

There is a need to provide a method for producing the micro-fabricated device such as a microarray that overcomes, or at least ameliorates, one or more of the disadvantages described above.

### SUMMARY

According to a first aspect, there is provided a microarray comprising a plurality of active agents immobilized onto an array of porous nanostructures, wherein each nanostructure has a network of pores that extend throughout at least one dimension of the nanostructure.

Advantageously, due to the porous nature of the nanostructures, the nanostructures tend to cluster together such that their distal ends are spaced closer to each other relative to the respective proximal ends of adjacent nanostructures.

Advantageously, the nanostructures are unique in that the porous nature of the nanostructures provides probing sites that greatly enhanced the sensitivity of the microarray. Due to the porosity of the nanostructures, especially at the distal end, the inventors have found that the immobilization efficiency of the active agents to the nanostructure may be increased by more than 60 as compared to the immobilization efficiency of identical molecules onto a flat substrate not having any nanostructure thereon. This increase in the immobilization efficiency of the active agents may be attributed to an increased surface area contributed by the surface roughness/porosity of the nanostructure.

The inventors have also found that a greater density of active agents can be immobilized onto the nanostructures as compared to a flat substrate, to a substrate with a roughened surface or to a substrate having less porous nanostructures (as in the case of silver etched wires).

According to a second aspect, there is provided a method of forming a microarray comprising the step of immobilizing active agents to an array of porous nanostructures, wherein each nanostructure has a network of pores that extend throughout at least one dimension of said nanostructure.

The method may be combined with conventional lithography techniques in order to form a microarray with a plurality of detection regions (or testing sites) in which the detection regions are separated from each other by substrate banks. As such, the size and position of each detection region can be controlled or determined by the use of a photoresist mask.

In the above microarray, each detection region comprises an array of porous nanostructures on the substrate. The various detection regions can be spaced apart from each other on the substrate. Each detection region can be used to test for the presence or absence of a specific target, or the amount of targets. Hence, the type of active agents in each detection region can be different or can be the same but at different concentrations. In this manner, in a situation where a number of targets in a sample are to be identified, the sample can be placed in contact with the microarray such that concurrent identification of the different types of targets can be carried out due to the different types of active agents present in the various detection regions. Accordingly, due to the ability to spatially determine the size and position of the various detection regions, thousands of detection regions can be fabricated onto the microarray (or chip). Hence, the disclosed method can be used to easily scale up a microarray.

More advantageously, the disclosed method may not require the use of complex lithography and etching techniques such as electron-beam lithography or reactive ion etching.

Furthermore, the disclosed method mitigates the problem of limited accessibility of targets and unfavorable interaction between intermediary linkers and substrate by mixing an intermediary linker and a target in a homogenous phase first to form a complex thereof, and then this complex to specific locations of the microarray.

According to a third aspect, there is provided a system for detecting the presence or absence of a target in a sample, comprising:

a microarray comprising a plurality of active agents immobilized onto an array of porous nanostructures, wherein each nanostructure has a network of pores that extends throughout at least one dimension of the nanostructure, and wherein the active agents have an affinity for the target and are coupled to a label to produce a signal when bound to the target; and a detector for detecting the signal produced by the label to determine the presence or absence of the target in the sample.

According to a fourth aspect, there is provided a microfluidic device for detecting the presence or absence of a target in a sample, comprising:

a microarray comprising a plurality of active agents immobilized onto an array of porous nanostructures, wherein each nanostructure has a network of pores that extends throughout at least one dimension of the nanostructure, and wherein the active agents have an affinity for the target and are coupled to a label to produce a signal when bound to the target;

a channel for directing the sample flow towards the microarray; and

a detector for detecting the signal produced by the label to determine the presence or absence of the target in the sample.

According to a fifth aspect, there is provided a method of making a microarray comprising the steps of:

contacting part of the area of an etchable substrate with catalyst particles that promote the rate of etching when the substrate is exposed to an etchant while leaving the remainder of the area of the substrate not exposed to the catalyst particles;

etching the substrate in the presence of an etchant to form porous nanostructures thereon from areas of the substrate that are not exposed to the catalyst particles, wherein each nanostructure has a network of pores that extends throughout at least one dimension of the nanostructure;

removing the etchant from the substrate to form an array of porous nanostructures on the substrate; and

immobilizing active agents to the array of nanostructure clusters.

### DEFINITIONS

The following words and terms used herein shall have the meaning indicated:

The terms “microarray” or “array” as used herein refers to an array of porous nanostructures on a substrate, wherein each porous nanostructure has a plurality of binding sites or probe sites that allow one or more active agents to be disposed therein.

The term “active agent” refers to any chemical molecule that is chemically active or biological agent that is biologically active. The active agent is capable of binding or reacting with a target or an intermediary bound to the target. The active agent may exhibit chemical activity or may exhibit biological activity. Exemplary active agent include proteins, antibodies, oligopeptides, small organic molecules, coordination complexes, aptamers, cells, cell fragments, virus particles, antigens, polysaccharides, lipids and polynucleotides, or combinations thereof. The active agents may be immobilized to or attached to the porous nanostructures.

The term “target” or “target analyte” refers to a substance to be detected that is capable of binding to or reacting with the active agent. The target may be a biological target or a chemical target. A biological target may also be a substance to be detected for calibration purposes. Exemplary biological targets include, but are not limited to, nucleic acids (such as DNA, RNA, nucleotides, or nucleosides), oligonucleotides, polynucleotides, drugs, hormones, proteins, enzymes, antibodies, carbohydrates, receptors, bacteria, cells, virus particles, spores, lipids, allergens and antigens. Exemplary chemical targets include, but are not limited to, an environmental contaminant such as organic materials (for example, aliphatic hydrocarbon compounds, aromatic-containing compounds and chlorinated compounds) or inorganic materials (for example, metals and nitrates), a chemical warfare agent such as nerve agents (for example, sarin, soman, tabun and cyclosarin), blood agents (for example, arsines and hydrogen cyanide), or lachrymatory agents (for example, tear gas and pepper spray), a herbicide, a pesticide, a chemical catalyst, or another chemical reactant of a chemical reaction. The target may bind or react directly with the active agent or may interact indirectly with the active agent through an intermediary linker. The target may be directly or indirectly coupled with a label to generate a signal. Typical labels include, but are not limited to, fluorescent labels, dyes, quantum dots, particles, enzymes, electrochemical active compounds or other signal generation entities.

The term “intermediary linker” refers to a moiety that is capable of connecting or coupling two or more moieties such as an active agent and a target together.

The intermediary linker may be made up of at least two structural units that are able to interact, immobilize or bind to the two or more moieties. The type of structural units making up the intermediary linker is then dependent on the type of active agent and target. For example, where the active agent is a single stranded sense oligonucleotide and the target is an antigen, the intermediary linker may be a moiety that is made up of two structural units of a single stranded anti-sense oligonucleotide and an antibody. Hence, the anti-sense oligonucleotide unit of the intermediary linker hybridizes with the sense oligonucleotide while the antibody unit of the intermediary linker binds with the antigen. In this manner, the intermediary linker serves to allow capturing of the targets by the active agents, which would not typically occur in the absence of such intermediary linkers since the target and active agents are not able to interact together.

The term “polynucleotide”, as used interchangeably with the term “nucleic acid”, is to be interpreted broadly to refer to a string of at least two base-sugar phosphate combinations. This term includes deoxyribonucleic acid (DNA), such as cDNA or genomic DNA, and ribonucleic acid (RNA), such as tRNA, snRNA, rRNA, mRNA, anti-sense RNA, RNAi, siRNA or ribozymes. The DNA or RNA may be unmodified DNA or RNA or may be modified DNA or RNA. The polynucleotide may include single- and double-stranded DNA, or mixture thereof, single- and double-stranded RNA, or mixture thereof, hybrid molecules comprising DNA and RNA that may be single-stranded or double-stranded, or a mixture thereof. The term polynucleotide also includes locked nucleic acids, peptide nucleic acids and analogues of RNA and DNA which do not occur naturally. An example of an artificial polynucleotide is L-DNA.

The terms “peptide”, “polypeptide” and “protein” are to be interpreted broadly to include linear molecular chains of amino acids, including fragments of single chain proteins. The peptide, polypeptide or protein can be isolated from nature or may be of viral, bacterial, plant or animal origin. The peptide, polypeptide or protein may be a synthetic peptide, polypeptide or protein. The peptide, polypeptide or protein may also refer to a naturally modified peptide, polypeptide or protein where the modification is effected, for example, by glycosylation, acetylation, phosphorylation and similar modifications which are well known in the art.

The term “affinity” can include biological interactions and/or chemical interactions. The biological interactions can include, but are not limited to, bonding or hybridization among one or more biological functional groups located on the active agent and the biological target. In this regard, the active agent can include one or more biological functional groups that selectively interact with corresponding biological functional groups on the biological target. The chemical interaction can include, but is not limited to, bonding (e.g., covalent bonding, ionic bonding, and the like) among one or more functional groups (e.g., organic and/or inorganic functional groups) located on the active agent and target.

The term “hybridize” and grammatical variants thereof, is to be interpreted broadly to refer to the pairing of a nucleic acid molecule to a complementary strand of this nucleic acid molecule to thereby form a hybrid. The hybridization can include complete hybridization (when all of the base pairs of both strands of nucleic acid molecules hybridize together) as well as partial hybridization (when the majority of the base pairs of both strands of nucleic acid molecules hybridize together). As such, these nucleic acid molecules are termed as “complementary” if they naturally bind to each other by base-pairing.

The word “substantially” does not exclude “completely” e.g. a composition which is “substantially free” from Y may be completely free from Y. Where necessary, the word “substantially” may be omitted from the definition of the invention.

Unless specified otherwise, the terms “comprising” and “comprise”, and grammatical variants thereof, are intended to represent “open” or “inclusive” language such that they include recited elements but also permit inclusion of additional, unrecited elements.

As used herein, the term “about”, in the context of concentrations of components of the formulations, typically means +/−5% of the stated value, more typically +/−4% of the stated value, more typically +/−3% of the stated value, more typically, +/−2% of the stated value, even more typically +/−1% of the stated value, and even more typically +/−0.5% of the stated value.

Throughout this disclosure, certain embodiments may be disclosed in a range format. It should be understood that the description in range format is merely for convenience and brevity and should not be construed as an inflexible limitation on the scope of the disclosed ranges. Accordingly, the description of a range should be considered to have specifically disclosed all the possible sub-ranges as well as individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Certain embodiments may also be described broadly and generically herein. Each of the narrower species and subgeneric groupings falling within the generic disclosure also form part of the disclosure. This includes the generic description of the embodiments with a proviso or negative limitation removing any subject matter from the genus, regardless of whether or not the excised material is specifically recited herein.

### DETAILED DISCLOSURE OF EMBODIMENTS

Exemplary, non-limiting embodiments of a microarray and a method for forming the same will now be disclosed.

The microarray comprises a plurality of active agents immobilized onto an array of porous nanostructures, wherein each nanostructure has a network of pores that extends throughout at least one dimension of said nanostructure.

The method comprises the step of immobilizing active agents to an array of porous nanostructures, wherein each nanostructure has a network of pores that extends throughout at least one dimension of the nanostructure.

The method may comprise the step of forming nanostructure clusters from the porous nanostructures. The porous nanostructure may have a proximal end extending from the substrate and a distal end opposite the proximal end. Accordingly, the nanostructure clusters may be made up of a plurality of nanostructures in which their distal ends are spaced closer to each other relative to the respective proximal ends of adjacent nanostructures.

The method may also comprise the steps of contacting part of the area of an etchable substrate with catalyst particles that promote the rate of etching when the substrate is exposed to an etchant while leaving the remainder of the area of the substrate not exposed to the catalyst particles; etching the substrate in the presence of an etchant to form porous nanostructures thereon from areas of the substrate that are not exposed to the catalyst particles, wherein each nanostructure has a network of pores that extends throughout at least one dimension of the nanostructure; removing the etchant from the substrate to form an array of porous nanostructures on the substrate and immobilizing active agents to the array of porous nanostructures. The removing step may result in the formation of nanostructure clusters from the array of porous nanostructures, wherein in each nanostructure cluster, the distal ends of the porous nanostructures are spaced closer to each other relative to the respective proximal ends of adjacent nanostructures.

The disclosed method may comprise the step of providing an array of porous nanostructures on a substrate. The providing step may comprise the step of selectively etching the substrate in order to fabricate the porous nanostructures. The nanostructures may be fabricated by metal-assisted catalytic etching (MACE) of the substrate in an etching solution with the aid of catalyst particles, such as metal nanoparticles, that may be deposited on the substrate by means of an oblique-angle deposition (also known as glancing-angle deposition or GLAD) technique. Hence, the providing step may comprise the step of forming the porous nanostructures on the substrate using a glancing angle deposition technique. This may involve contacting part of the area of the substrate with a plurality of catalyst particles that promote the rate of etching when the substrate is exposed to an etchant while leaving the remainder of the area of the substrate not exposed to the catalyst particles. This combination of GLAD and MACE techniques is hereby termed as “GLAD-MACE”. The metal nanoparticles may act as catalysts in the etching of the substrate beneath them. Thus, when subjected to MACE, the substrate surface in contact with the catalyst particles is catalytically etched away. As a result, nanostructures may be formed from the substrate surface which is not in contact with the catalyst particles.

The method may comprise the step of, after the etching step, drying the porous nanostructures to thereby cause the porous nanostructures to cluster together to form a nanostructure cluster, wherein in each nanostructure cluster, the distal ends of the porous nanostructures are spaced closer to each other relative to the respective proximal ends of adjacent nanostructures.

After the porous nanostructures or nanostructure clusters are formed on the substrate, the method may comprise the step of immobilizing the active agents onto the porous nanostructures or nanostructure clusters.

The array of nanostructures may be disposed on a substrate. The substrate may be glass, carbon, silicon (Si), SiGe, GaN, SiC and GaAs. For the carbon based substrate, plasma etching using argon and/or oxygen as the etching gases can be used. In one embodiment, the substrate is silicon.

The disclosed method may comprise the following steps. The substrate may be cleaned in order to remove any impurities that may interfere with the subsequent steps.

The substrates may then be subjected to an etching step in an acidic solution prior to the GLAD step in order to remove any native materials (such as native oxides) that may be present. The etching step may be carried out for a period selected from the group consisting of about 30 seconds to about 5 minutes, about 1 minute to about 5 minutes, about 2 minutes to about 5 minutes, about 3 minutes to about 5 minutes, about 4 minutes to about 5 minutes, about 30 seconds to about 1 minute, about 30 seconds to about 2 minutes, about 30 seconds to about 3 minutes and about 30 seconds to about 4 minutes. In one embodiment, the etching step may be carried out for about 1 minute when HF is used as the acidic solution.

The GLAD step should be carried out under conditions in which the vapor flux arrives at the substrate in approximately a straight line. For this reason, this step is preferably carried out under conditions approximating a vacuum, at a pressure less than 10⁻⁵ torr, or less than 10⁻⁶ torr. In order to achieve this pressure, the GLAD step may be carried out in an electron beam evaporator. At higher pressures, scattering from gas molecules present in the evaporator tends to prevent well defined nanoparticles from growing.

The substrate normal may be placed at an angle selected from the range of about 85° to about 90°, about 85° to about 86°, about 85° to about 87°, about 85° to about 88°, about 85° to about 89°, about 86° to about 90°, about 87° to about 90°, about 88° to about 90° and about 89° to about 90° to the direction of the incoming flux. In one embodiment, the angle may be about 87°. It is to be noted that the angle of deposition should be chosen to allow the deposit of discrete catalyst particles and not a film of catalyst particles. Accordingly, a deposition angle of less than about 80° should be avoided.

The substrate may be rotated at a rate selected from the group consisting of about 0.01 rpm to about 10 rpm, about 0.1 rpm to about 1 rpm, about 0.5 rpm to about 1 rpm and about 0.1 rpm to about 0.3 rpm. In one embodiment, the rotational rate of the substrate may be about 0.2 rpm.

The catalyst particles are not particularly limited and exemplary catalyst particles may be selected from the group consisting of Au, Pt, Pd and Cu. It is to be appreciated that any metal catalysts that can be used in the GLAD-MACE technique are included. In one embodiment, the catalyst particles are Au nanoparticles. The etching solution may comprise water, HF and an oxidizing agent which may be selected from, but not limited to, H₂O₂, AgNO₃, KMnO₄ and Fe(NO₃)₃. In one embodiment, H₂O₂ is used.

In one embodiment, gold (Au) nanoparticles may be deposited on a Si substrate via GLAD and used as catalysts in the MACE step to etch silicon (Si) with an etching solution comprising of H₂O, H₂O₂ and HF. The Au nanoparticles may facilitate the reduction of H₂O₂, resulting in the generation of holes, which get injected into the Si via the Au nanoparticles. This injection of holes in turn may facilitate the etching of Si by HF. Hence, the Si in the vicinity of the Au nanoparticles may be etched away, causing a collective sinking of the Au nanoparticles into the Si. As a result of the dense network of Au nanoparticles on Si generated by the GLAD step and the sinking of the Au nanoparticles into the Si, freestanding nanostructures remain after the GLAD MACE step.

In the disclosed method, the duration of the GLAD step may be in the range selected from the group consisting of about 15 minutes to about 200 minutes, about 15 minutes to about 90 minutes and about 30 minutes to about 90 minutes. In one embodiment, the duration of the GLAD step may be about 30 minutes, or about 90 minutes. It is to be noted that the longer the duration of the GLAD step, more and bigger catalyst particles may be deposited on the substrate. Due to the different amount and size of the catalyst particles deposited, the porosity, particle size distribution and extent of clustering of the resultant nanostructures may be altered or substantially controlled.

The catalyst particles may be deposited as discrete particles, rather than a continuous thin film of catalyst particles. Hence, the diameter (if the catalyst particles are substantially spherical) or equivalent diameter (if the catalyst particles are substantially non-spherical) of the catalyst particles deposited may be selected from the group consisting of about 1 nm to about 100 nm, about 20 nm to about 100 nm, about 40 nm to about 100 nm, about 60 nm to about 100 nm, about 80 nm to about 100 nm, about 1 nm to about 20 nm, about 1 nm to about 40 nm, about 1 nm to about 60 nm, about 1 nm to about 80 nm, about 20 nm to about 40 nm, about 30 nm to about 40 nm, about 1 nm to about 3 nm and about 11 nm to about 13 nm. In one embodiment, the diameter of the catalyst particles is about 3 nm or about 12 nm. The dimensions of the catalyst particles may be equal to each other or may be different.

The method may comprise, after the GLAD step, the step of catalytically etching the substrate. The duration of the catalytically etching step may be selected from the group consisting of about 1 minute to about 120 minutes, about 10 minutes to about 15 minutes, about 10 minutes to about 20 minutes, about 10 minutes to about 25 minutes, about 15 minutes to about 20 minutes, about 15 minutes to about 25 minutes and about 19 minutes to about 21 minutes. In one embodiment, the catalytically etching step or metal-assisted catalytically etching step is carried out for about 20 minutes.

The concentration of HF may be selected from the group of about 1 M to about 27 M, about 1 M to about 10 M, about 1 M to about 20 M, about 1 M to about 25 M, about 10 M to about 27 M, about 15 M to about 27 M, about 25 M to about 27 M, about 4 M to about 5 M and about 4.5 M to about 4.7 M. In one embodiment, the concentration of HF is about 4.6 M.

The concentration of H₂O₂ may be selected from the group of about 0.2 M to about 9.8 M, about 0.2 M to about 2 M, about 0.2 M to about 4 M, about 0.2 M to about 6 M, about 0.2 M to about 8 M, about 2 M to about 9.8 M, about 4 M to about 9.8, about 6 M to about 9.8 M, about 8 M to about 9.8 M, and 0.43 M to about 0.45M. In one embodiment, the concentration of H₂O₂ may be about 0.44 M.

It is to be noted that the concentrations of the etching agents may be modified in order to adjust the height of the nanostructures, size of the clusters (leading to a change in the morphology of the resultant clusters) or size of the pores present in the nanostructures.

The temperature used during the MACE step may be from room temperature (or about 20° C. to about 25° C.) to about 50° C., from about 30° C. to about 50° C., from about 40° C. to about 50° C., from about 20° C. to about 30° C. and from about 20° C. to about 40° C.

After the MACE step, nanostructures may be viewed on the surface of the substrate. The nanostructures may be nanocolumns, nanopillars or nanowires. In one embodiment, the nanostructures are nanowires.

The nanostructures may have a height dimension that is longer than any other dimension, such as width or breadth of the nanostructure. The nanostructures typically extend from the substrate from a proximal end to a distal end, wherein the height dimension extends between said proximal and distal ends.

The thickness of the nanostructures (as defined by the width and/or breadth dimension) may be selected from the group consisting of about 1 nm to about 500 nm, about 1 nm to about 10 nm, about 1 nm to about 100 nm, about 1 nm to about 200 nm, about 1 nm to about 300 nm, about 1 nm to about 400 nm, about 10 nm to about 500 nm, about 100 nm to about 500 nm, about 200 nm to about 500 nm, about 300 nm to about 500 nm, about 400 nm to about 500 nm and about nm to about 100 nm. In an embodiment where the nanostructure is substantially cylindrical-shaped, the thickness may refer to the diameter of the nanostructure.

The height of the nanostructures (which is the distance between the proximal and distal ends of the nanostructure) may be selected from the group consisting of from about 1 μm to about 100 μm, about 1 μm to about 12 μm, about 1 μm to about 15 μm, about 1 μm to about 2 μm, about 1 μm to about 3 μm, and about 11 μm to about 13 μm. In one embodiment, the height of the nanostructures may be about 1 μm, about 3 μm or about 12 μm.

The density of the nanostructures per unit area may be in the range of about 1×10⁶ mm⁻² to about 2.5×10¹¹ mm⁻², about 1×10⁶ mm⁻² to about 1×10¹⁰ mm⁻², about 1×10⁷ mm⁻² to about 1×10¹⁰ mm⁻², about 1×10⁸ mm⁻² to about 1×10¹⁰ mm⁻², about 1×10⁹ mm⁻² to about 1×10¹⁰ mm⁻², about 1×10⁶ mm⁻² to about 1×10⁷ mm⁻², about 1×10⁶ mm⁻² to about 1×10⁸ mm⁻², about 1×10⁶ mm⁻² to about 1×10⁹ mm⁻², about 2×10⁷ mm⁻² to about 1×10¹⁰ mm⁻², about 2×10⁹ mm⁻² to about 1×10¹⁰ mm⁻² and about 2.5×10⁷ mm⁻² to about 2.5×10⁹ mm⁻².

The distance between each nanostructure may be equal or may vary.

More than one porous nanostructure may come towards each other and cluster together, typically at the ends of the nanostructures. The nanostructures may cluster together at the distal ends of the nanostructures due to the higher porosity at the distal ends compared to the proximal ends. The distal ends are more porous than the proximal ends since the distal ends are subjected to a longer etching time than the proximal ends. Accordingly, the nanostructure may form clusters in which the distal ends of the nanostructures are spaced closer to each other relative to the respective proximal ends of adjacent nanostructures.

The size of the pores extending through the nanostructures may be in the range of about 0.1 nm to about 10 nm. In embodiments where the pores can be viewed as having a substantially circular cross-sectional area, the above dimension can refer to the diameter of the pores. In one embodiment, the pores of said network of pores extend throughout a dimension of the nanostructure that excludes the height dimension.

In one embodiment, the nanostructures have a width and breadth dimension that are less than the height dimension and wherein the height dimension extends along a longitudinal axis extending between the proximal end to the distal end, and wherein the pores of said network of pores extend throughout the width dimension which is normal to the longitudinal axis. The width dimension and breadth dimension may be the same or different.

The pores may extend throughout the nanostructure such that the nanostructure may be viewed as having a network of pores across not only a selected height of the nanostructure, but also across the width and/or breadth of the nanostructure. The pores may be randomly distributed pores and may extend into the nanostructure at various orientations. The pores can be viewed as penetrating throughout the width and/or breadth of the nanostructure. The pores can be viewed as penetrating throughout the thickness of the nanostructure. The pores may not be limited to the surface of the nanostructure. Hence, the nanostructure may not be made up of a dense (non-porous) core surrounded by a porous shell. As such, the nanostructure may not have a core-shell configuration. The distal ends of the nanostructures may be more porous than the proximal ends such that the porosity of the distal ends.

The size of the clusters may vary from each other and may be in the micro-size range. For example, the size of the clusters may be in the range of about 1 μm to about 5 μm. The distance between each cluster may be selected from the group consisting of about 100 nm to about 10 μm, about 100 nm to about 500 nm, about 500 nm to about 1 μm, about 1 μm to about 5 μm, about 5 μm to about 10 μm, about 100 nm to about 1 μm, about 1 μm to about 10 μm, about 500 nm to about 5 μm and about 500 μm to about 10 μm.

The surface area of the nanostructure clusters (as defined by the perimeter per unit area) may be modified by controlling the extent of clustering of the nanostructures. The extent of clustering may be controlled by drying the substrate in different media after the GLAD-MACE step. For example, the substrate may be dried in de-ionized water, alcohol (such as methanol, ethanol, 2-propanol or butanol) or de-ionized water with N₂ flow (that is, dried with a nitrogen gun). As such, the surface area of the nanostructure clusters may be in the range of about 1 μm⁻¹ to about 3 μm⁻¹, about 1.5 μm⁻¹ to about 3 μm⁻¹, about 2 μm⁻¹ to about 3 μm⁻¹, about 2.5 μm⁻¹ to about 3 μm⁻¹, about 1 μm⁻¹ to about 1.5 μm⁻¹, about 1 μm⁻¹ to about 2 μm⁻¹, about 1 μm⁻¹ to about 2.5 μm⁻¹, about 1.5 μm⁻¹ to about 2 μm⁻¹, about 2 μm⁻¹ to about 2.5 μm⁻¹, about 1.8 μm⁻¹ to about 1.9 μm⁻¹, about 1.9 μm⁻¹ to about 2 μm⁻¹ and about 2 μm⁻¹ to about 2.1 μm⁻¹.

Advantageously, the use of different liquid media allows the degree of clustering of the nanostructures to be tuned in order to obtain different morphologies or surface area of the clusters. This may be achieved by varying the rate of removal of the liquid medium. For example, a slower rate of removal of the liquid medium (which depends on the volatility of the liquid medium) will result in smaller clusters being formed while conversely a more rapid rate of liquid medium removal will result in larger clusters forming. For example, water tends to form smaller clusters relative to more volatile media such as alcohols due to the slower rate of evaporation at the same temperature and pressure. The temperature and pressure at which the liquid medium is removed may also be altered.

The substrates are typically dried until the liquid media evaporates substantially completely. Typically, the substrate is left to dry overnight.

The catalyst particles on the substrate may then be removed using standard commercially available etchants.

The nanostructures on the substrate may be subjected to an oxidizing step. Hence, the method may comprise the step of, before the functionalizing step, oxidizing the nanostructures. The oxidizing step may be undertaken in an oxygen atmosphere at a certain temperature and time. The oxidizing temperature may be selected from the range of about 850° C. to about 950° C., or about 900° C. The oxidizing time may be selected from the range of about 30 minutes to about 40 minutes, or about 35 minutes. It is to be appreciated that the oxidizing temperature and oxidizing time is not particularly limited to that described above but can be of any temperature and time that are sufficient for the nanostructures and exposed surfaces of the substrate to be oxidized.

The disclosed method may comprise the step of immobilizing active agents to the array of porous nanostructures or nanostructure clusters.

The active agent may be a polynucleotide. The active agent may have an affinity for a biological target. The polynucleotide may be a single stranded oligonucleotide. The single stranded oligonucleotide may be complementary to a target oligonucleotide. Hence, the single stranded oligonucleotide may be termed as a single stranded sense oligonucleotide while the target oligonucleotide may be termed as a single stranded anti-sense oligonucleotide. The active agent may be coupled with a label to give off a signal. The label is not particularly limited and may include any label that is known to a person skilled in the art. An exemplary label may be a fluorescent dye such as cyanine 3 (Cy 3) or cyanine 5 (Cy 5) such that the signal given off by the active agent is a fluorescent signal. The label may be emitted only when the active agent binds to the target. Alternatively, the active agent may be emitting a signal that is quenched when the active agent binds to the target. Other detection methods can include measurement of the change in electrical conductance, radioactivity, enzymatic reaction, or chemi-luminescence. It is to be appreciated that the type of detection methods that can be used are not limited to the above and that the person skilled in the art would know what type of detection method to use based on the target to be analyzed.

The concentration of the active agents on the substrate may be in the range of about picomolar to about micromolar.

The density of the active agents on the substrate may be in the range of about 1×10³ mm⁻² to about 1×10¹⁸ mm⁻², about 1×10³ mm⁻² to about 1×10⁶ mm⁻², about 1×10³ mm⁻² to about 1×10⁹ mm⁻², about 1×10³ mm⁻² to about 1×10¹² mm⁻², about 1×10³ mm⁻² to about 1×10¹⁵ mm⁻², about 1×10⁵ mm⁻² to about 1×10¹⁸ mm⁻², about 1×10⁹ mm⁻² to about 1×10¹⁸ mm⁻², about 1×10¹² mm⁻² to about 1×10¹⁸ mm⁻² and about 1×10¹⁵ mm⁻² to about 1×10¹⁸ mm⁻².

The immobilization efficiency of the active agents to the nanostructure clusters may be increased by at least about 60 folds as compared to the immobilization efficiency of identical active agents onto a flat substrate not having any porous nanostructures thereon. The immobilization efficiency may be increased by at least about 100 folds, about 150 folds, about 200 folds, about 250 folds, about 300 folds, about 350 folds, about 400 folds, about 450 folds or about 500 folds as compared to the immobilization efficiency of identical active agents onto a flat substrate not having any, nanostructure clusters thereon. Without being bound by theory, the inventors believe that the immobilization efficiency of the active agents to the porous nanostructures can be increased as compared to the immobilization efficiency of identical active agents on other types of surfaces or nanostructures (that do not cluster together) due to one of increased porosity and/or increased surface area of the nanostructure clusters.

Furthermore, the hydrophilicity of the substrate facilitates the penetration of a biological fluid acting as medium for the active agent or intermediary linker, thus improving the immobilization of the active agents to the porous nanostructures and the resultant binding of the intermediary linker to the active agents.

The amount of active agents that can be immobilized onto the porous nanostructures or nanostructure clusters, is not limited and can be defined by the signal-to-noise ratio which is typically at least 200, at least 210, at least 220, at least 230, at least 240, at least 250, at least 260, at least 270, at least 280, at least 290, at least 300, at least 350, at least 400, at least 450 or at least 500. In one embodiment, where the loading concentration of a sense oligonucleotide was 20 μM, the signal-to-noise ratio was 204. In another embodiment, where the loading concentration of a sense oligonucleotide was 1 μM, the signal-to-noise ratio was 280. Hence, the disclosed microarray can be used to immobilize active agents with a high signal-to-noise ratio.

The immobilizing step may comprise the step of functionalizing the surfaces of the nanostructure cluster with a linker molecule. In an embodiment where the active agent is a single stranded oligonucleotide, the functionalizing step may comprise the step of forming amine groups on the surface of the nanostructure clusters. Hence, the linker molecule may have a functional group that is capable of bonding to the surface of the nanostructures as well as an amine functional group. After the surfaces are aminated, the method may comprise the step of carboxylating the surface of the nanostructures. Here, the linker molecule is one that may have a functional group that is able to react with the amine groups present on the surface of the nanostructures as well as a carboxyl functional group. It should be noted that other chemical reactions can be employed to functionalize the nanostructures in order to immobilize the active agent, which is then dependent on the type of active agent.

Due to the presence of the carboxyl groups on the surfaces of the nanostructure clusters, the active agents may be immobilized or coupled onto the surfaces of the nanostructure clusters. As mentioned above, other reactive groups may be used to immobilize or couple the active agents onto the porous nanostructures or nanostructure clusters.

In one embodiment, the nanostructures are comprised of silicon that is oxidized to form a SiO₂ layer on the surface. Here, (3-Aminopropyl)triethoxysilane was used as a linker molecule to form the amine functional groups on the surface. Following which, the nanostructures are carboxylated with succinic anhydride and the active agent (a sense-strand oligonucleotide with 5′-amino and 3′-Cy3 modifications) was coupled to the carboxyl-terminated surface using 1-Ethyl-3-(3-dimethylaminopropyl)carbodiimide (EDC) and 1-hydroxy-2-nitro-4-benzenesulfonic acid (HNSA).

The active agent may be used to detect the presence or absence or amount of a target in a sample. The target may be coupled to a label to give off a signal. The label may be a fluorescent label as mentioned above. The label may be different from that used on the active agent. The label may only emit a signal when the active agent binds with the target or alternatively the label may be emitting a signal that is quenched when the active agent binds with the target. In other embodiments, for example, the target may not be coupled with a label. Instead, a second recognition molecule with a label can be used, or a second recognition molecule that can react with the target molecule to give off a signal can be used.

For a DNA microarray, the target may be a biological target such as a single stranded antisense oligonucleotide.

For a protein microarray, the target may be a biological target such as an analyte (for example, an antigen) that has an affinity for a polypeptide (such as an antibody). The analyte may be captured by an intermediary linker (that is, an antibody-antisense conjugate) and then bound to the porous nanostructures or nanostructure clusters by the hybridization between the sense oligonucleotide (the active agent) and antisense oligonucleotide (of the intermediary linker). In this manner, the analyte can be captured by the active agent and this (indirect) interaction between the analyte and the active agent can be determined by the signal given off. Advantageously, the analyte can be captured by the intermediary linker in a liquid phase (homogeneous phase) first and then this analyte-intermediary linker moiety can be detected by the microarray due to the hybridization effects between the oligonucleotides of the intermediary linker and active agent.

The microarray can also be used to test for the presence of a chemical contaminant (the target analyte). Here, the active agent may be a chemical agent that can give off a detectable signal upon reaction with the chemical contaminant.

The microarray can also be used as reaction sites for a chemical reaction. Here, the active agent may be one chemical reactant of a desired reaction, which upon contact with another chemical reactant (the target analyte) present in a test sample, reacts together. The reaction can be detected by one of the detection methods disclosed above.

The method may be combined with conventional lithography techniques in order to form a microarray with a plurality of detection regions (or testing sites) in which the detection regions are separated from each other by substrate banks. Hence, the method may comprise the step of forming a plurality of detection regions on the substrate, each detection region comprising active agents immobilized to the array of porous nanostructures, wherein each detection region comprises a specific type of active agents that are the same as or different between the detection regions. The method may comprise the step of providing a photoresist with openings having a desired shape and dimension on a substrate. The substrate having the photoresist thereon may be subjected to lithography such as photolithography. Hence, the area of the substrate that is covered by the photoresist forms the substrate banks while the areas of the substrate that are not in contact with the photoresist form the detection regions.

Alternatively, other methods can be used to form the detection regions. For example, active agents can be grafted onto specific regions of the substrate by activation through light. In addition, Dip-Pen like technology can be employed to deliver and graft active agents to specific locations.

The size and position of each detection region can be controlled or determined by the use of a photoresist mask. The shape and dimension of the detection regions are not particularly limited and can be chosen based on the needs of the user.

The detection regions can be formed before or after the GLAD-MACE step.

After the porous nanostructures or nanostructure clusters as well as the detection regions have been formed, the substrate may be subjected to a removal step. After removal of the metal catalysts, the substrate may then be oxidized to form a microarray having a plurality of detection regions. The porous nanostructure or nanostructure clusters in each detection region can be subjected to the above functionalization steps in order to bind desired active agents to the surfaces of the porous nanostructure or nanostructure cluster which can then be used to detect desired targets. Alternatively, other functionalization steps can be used depending on the type of active agent to be immobilized or attached to the porous nanostructure or nanostructure clusters.

As mentioned above, each detection region can be specific for one type of target since different active agents can be immobilized to the porous nanostructure or nanostructure clusters present in each detection region. Due to the spatial separation of each detection region on the microarray, the detection regions can be treated independently of each other so that one type of active agent can be present in each detection region. Alternatively, more than one active agent can be immobilized to the porous nanostructures or nanostructure clusters present in the same detection region. The different active agents and their associated targets (or reactions) can be detected using different types of detection methods or detection labels.

This microarray can be used to test for the presence, the amount or to identify a plurality of targets in a sample. The sample can be placed in contact with the microarray such that concurrent identification of the different types of targets present in the sample can be carried out due to the different types of active agents present in the various detection regions or in some instances, in the same detection regions.

This type of microarray is also termed as an analyte-specific spatially addressable nanostructured array (ASANA) in the following section.

Advantageously, the disclosure microarray can be produced in a large area, highly scalable platform. The disclosed platform can be used to contain many active agents (or termed in the following sections as analyte-specific reagents, or ASR) to allow molecular recognition of specific molecules or reactions of interest.

More advantageously, the nanostructure clusters may have a superhydrophilic effect that allows for extreme wettability in the presence of biological buffers. Hence, this may promote the interaction between the targets that may be present in the buffers with the active agents immobilized on the nanostructure clusters.

Advantageously, the intermediary linker and target may be mixed in the solution first to form a conjugate. The conjugate may then be immobilized to the active agent present on the array. Hence, by having the interaction between the target and intermediary linker in the homogenous phase, this may aid to mitigate the inaccessibility problem (between the active agents and the targets) and unfavorable interaction between the substrate and intermediary linker.

The microarray may be part of a system for detecting the presence of a target in a sample.

Hence, there is provided a system for detecting the presence or absence of a target in a sample, comprising a microarray comprising a plurality of active agents immobilized onto an array of porous nanostructures, wherein each nanostructure has a network of pores that extends throughout at least one dimension of the nanostructure, and wherein the active agents have an affinity for the target and are coupled to a label to produce a signal when bound to the target; and a detector for detecting the signal produced by the label to determine the presence or absence of the biological target in said sample.

The microarray may be part of a microfluidic device for detecting the presence of a target in a sample. Hence, there is provided a microfluidic device for detecting the presence or absence or amount of a target in a sample, comprising a microarray comprising a plurality of active agents immobilized onto an array of porous nanostructures, wherein each nanostructure has a network of pores that extend throughout at least one dimension of the nanostructure, and wherein the active agents have an affinity for the target and are coupled to a label to produce a signal when bound to the target; a channel for directing the sample flow towards the microarray; and a detector for detecting the signal produced by the label to determine the presence or absence of the target in the sample. The amount of the target in the sample can also be determined by the signal produced.

The microarray may be partitioned or cut to form individual detection regions. One or more detection regions can be formed as part of a microfluidic device. Accordingly, there is provided a microfluidic device for detecting the presence or absence or amount of a target in a sample, comprising a detection region comprising a plurality of active agents immobilized onto an array of porous nanostructures, wherein each nanostructure has a network of pores that extend throughout at least one dimension of the nanostructure, and wherein the active agents have an affinity for the target and are coupled to a label to produce a signal when bound to the target; a channel for directing the sample flow towards the detection region; and a detector for detecting the signal produced by the label to determine the presence or absence of the target in the sample. The amount of target present in the sample can also be determined by the signal produced.

Other auxiliary parts of the microfluidic device may include micropumps, valves and other flow-control microfluidic technologies, which would be apparent to a person skilled in the art when tailoring such microfluidic devices as needed.

The detector is not particularly limited and the person skilled in the art would know what type of detector to use based on the type of label used or type of analyte to be detected or quantify. Advantageously, the disclosed method may be entirely scalable over large areas (up to 4″ wafers or more) and may not require complex lithography (such as electron-beam lithography) and etching processes (such as deep-reactive ion etching).

### BRIEF DESCRIPTION OF DRAWINGS

The accompanying drawings illustrate a disclosed embodiment and serves to explain the principles of the disclosed embodiment. It is to be understood, however, that the drawings are designed for purposes of illustration only, and not as a definition of the limits of the invention.

FIG. 1 is a schematic diagram representing the basic structure of an Analyte-specific Spatially Addressable Nanostructured Array (ASANA).

FIG. 2 illustrates the process flow of functionalization of the nanostructures with DNA-probes and the binding of these probes to the analytes.

FIG. 3 shows the fabrication of the various substrates used in the examples, such as a flat substrate, an IL-CE substrate and a GLAD-MACE substrate.

FIG. 4(a) is a scanning electron microscope (SEM) image (with a scale bar of 1 μm) of a substrate with silicon nanostructures fabricated by interference lithography-chemical etching (IL-CE) using Au as catalyst.

FIG. 4(b) is a SEM image (with a scale bar of 1 μm) of a substrate with silicon nanostructures (having a height of 1 μm) fabricated by the disclosed GLAD-MACE method using Au as catalyst.

FIG. 4(c) is a SEM image (with a scale bar of 1 μm) of a substrate with silicon nanostructures (having a height of 3 μm) fabricated by the disclosed GLAD-MACE method using Au as catalyst.

FIG. 4(d) is a SEM image (with a scale bar of 2 μm) of a substrate with silicon nanostructures (having a height of 12 μm) fabricated by the disclosed GLAD-MACE method using Au as catalyst.

FIG. 4(e) is a SEM image (with a scale bar of 1 μm) of a substrate with silicon nanostructures (with a height of 12 μm) fabricated by the disclosed GLAD-MACE method using Ag as catalyst.

FIG. 4(f) is a graph showing the density of reactive amine group on various fabricated substrates via the relative fluorescent unit (RFU) readings of directly coupled Cy5 (1:100).

FIG. 4(g) is a graph showing the RFU readings between directly coupled Cy5 dilutions on flat and GLAD-MACE surfaces with different Cy5-NHS dilutions.

FIG. 5(a) is a SEM image (with a scale bar of 1 μm) showing silicon nanowires obtained from the IL-CE method.

FIG. 5(b) is a transmission electron microscopy (TEM) image (with a scale bar of 100 nm) showing the top section of a GLAD-MACE nanowire obtained with Au catalysts. The inset is a high-resolution transmission electron microscopy (HRTEM) image (with a scale bar of 20 nm) of the same.

FIG. 5(c) is a TEM image (with a scale bar of 100 nm) showing the bottom section of a GLAD-MACE nanowire obtained with Au catalysts. The inset is a HRTEM image (with a scale bar of 20 nm) of the same.

FIG. 5(d) is a TEM image (with a scale bar of 100 nm) showing the top section of a GLAD-MACE nanowire obtained with Ag catalysts. The inset is a HRTEM image (with a scale bar of 10 nm) of the same.

FIG. 5(e) is a TEM image (with a scale bar of 100 nm) showing the bottom section of a GLAD-MACE nanowire obtained with Ag catalysts. The inset is a HRTEM image (with a scale bar of 10 nm) of the same.

FIG. 6(a) is a SEM image (with a scale bar of 2 μm) showing a substrate with a roughened surface that was produced by catalytically etching the substrate with a thin Au film for 2 minutes.

FIG. 6(b) is a SEM image (with a scale bar of 2 μm) showing a substrate with a roughened surface that was produced by catalytically etching the substrate with a thin Au film for 20 minutes.

FIG. 6(c) is a graph showing the density of reactive amine groups on flat, thin metal-CE and GLAD-MACE nanostructured silicon surfaces generated via RFU readings of directly coupled Cy5 (1:100). Au was used as the metal catalyst.

FIG. 7(a) shows the process flow for fabricating an ASANA microarray based on the GLAD-MACE process.

FIG. 7(b) shows a picture showing the top-view of the ASANA microarray.

FIG. 8(a) shows the fluorescent intensity of the GLAD-MACE substrate as compared to flat silica substrate after coupling of various concentrations of Cy3 labeled single-strand DNA (ssDNA) oligonucleotides.

FIG. 8(b) shows the RFU readings of a sense oligonucleotide (Cy3) at various concentrations of NH₂-Cy3 labeled ssDNA oligonucleotides.

FIG. 9(a) shows the comparison of the loading density of sense and antisense ssDNA on GLAD-MACE microarray chip via the fluorescent intensity of Cy3 coupled sense strand and Cy5 coupled target strand on GLAD-MACE surfaces at various concentrations of Cy3 labeled ssDNA oligos and Cy5 ssDNA anti-sense oligo at 20 μM.

FIG. 9(b) is a graph showing the comparison of the loading density of sense and antisense ssDNA on GLAD-MACE microarray chip via the various RFU readings of Cy3 coupled sense strand and Cy5 coupled target strand on GLAD-MACE surfaces at various concentrations of Cy3 labeled ssDNA oligos and Cy5 ssDNA anti-sense oligo at 20 μM.

FIG. 10(a) shows the detection of protein analyte in human serum using ASANA array via the fluorescent signals on flat substrate and ASANA arrays with captured analytes.

FIG. 10(b) shows the comparison between normalized RFU (analyte/ASR, Cy5/Cy3) of the protein analyte in human serum detected using ASANA array.

FIG. 11 is a schematic diagram showing the use of an alternative ASANA platform based on optical activation of the analytes.

### DETAILED DESCRIPTION OF DRAWINGS

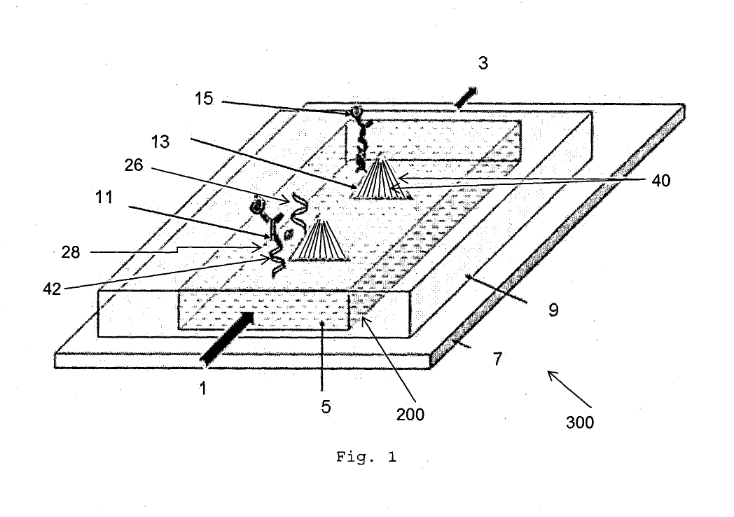

FIG. 1 is a schematic diagram representing the basic structure of an Analyte-specific Spatially Addressable Nanostructured Array (ASANA) 200. The ASANA 200 comprises a plurality of porous nanostructures 40 forming a nanostructure cluster 13. An active agent 26 such as sense DNA is immobilized on the surface of the nanostructure cluster 13, which hybridizes with intermediary linker 28 in order to detect the presence of or the absence of or the amount of a labeled analyte 15. The intermediary linker 28 is produced by conjugating an antibody 11 with an antisense DNA 42. The ASANA 200 is contained by a housing 9 which has a channel 5 therethrough for a sample to flow through. The housing 9 containing the ASANA 200 is placed on a slide 7 to form a microfluidic device 300. In use, a sample is flown through the channel 5 in the direction depicted by the arrow 1 towards arrow 3. If a target analyte 15 is present in the sample, the target analyte 15 binds to the intermediary linker 28. The complex of target 15 and intermediary linker 28 then binds to the active agent 26 via base pairing. The binding of the target analyte 15 to the intermediary linker 28 and subsequent immobilization to the active agent 26 can be detected by a detector (not shown) due to the label provided on the target analyte 15. Hence, FIG. 1 shows that the nanostructured ASANA 200 device can be integrated with microfluidics to allow for enhanced high-density capture of target analytes 15 by addressable DNA mediated assembly of analyte-specific reagents (ASR).

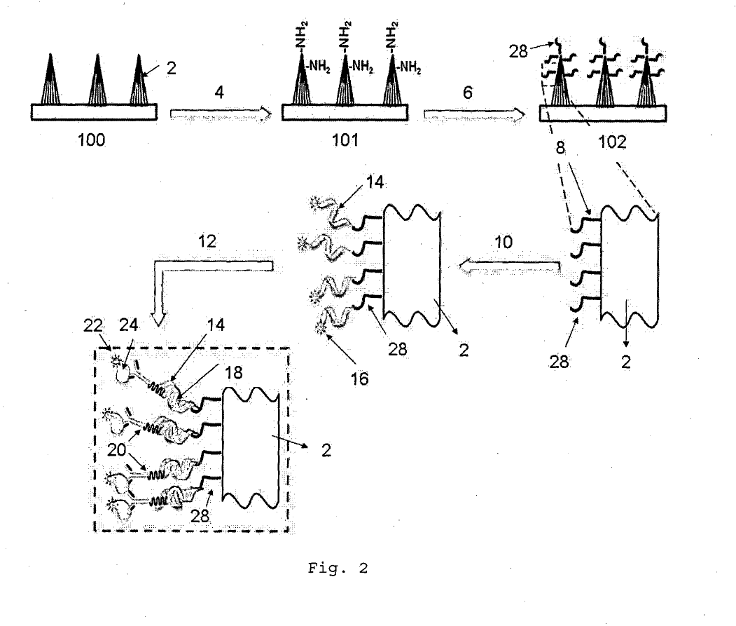

FIG. 2 depicts the process flow of functionalization of the nanostructures with an active agent such as a sense DNA and the binding of these active agents to the analytes. The nanostructure clusters 2 that are formed on the surface of a substrate by the GLAD-MACE technique are oxidized to form an oxidized GLAD substrate 100. The oxidized GLAD substrate 100 is subjected to an amination step 4 to cause amine groups to be present on the surface of the nanostructure clusters 2, thus forming an aminated GLAD substrate 101. The aminated GLAD substrate 101 is subjected to a carboxylation step 6 to cause carboxyl groups 28 to be present on the surface of the nanostructure clusters 2, thus forming a carboxylated GLAD substrate 102. The carboxylated GLAD substrate 102 is then subjected to an incubation step 10 by contacting the carboxylated GLAD substrate 102 with a solution of active agents 14 (that are coupled to a label 16). The active agents 14 are immobilized onto the surface of the nanostructure clusters 2 due to the 5′-amino groups of the active agents 14 with the carboxyl groups 28 present on the nanostructure clusters 2. The substrate is then exposed to a solution containing a target analyte 24. The target analyte 24 (coupled to another label 22) binds to an intermediary linker (made up of an analyte binding portion 20 and a active agent binding portion 18) when in solution (that is in the homogenous phase). The complex made up of the target analyte 24 and intermediary linker then hybridizes with the active agent 14 in a hybridization step 12 due to complementary base pairing between the active agent 14 and active agent binding portion 18 of the intermediary linker. The binding of the target analyte 24 to the substrate can be detected by the signal given off by the label 22. In this manner, the substrate can be used as a protein microarray.

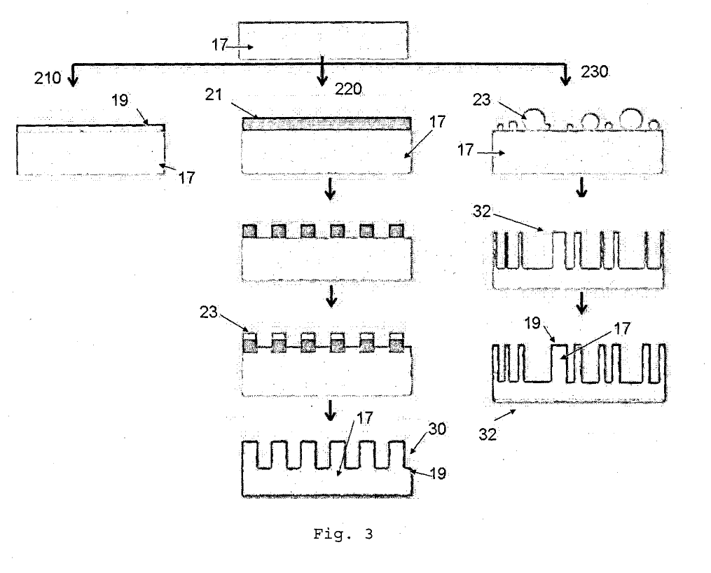

FIG. 3 depicts the fabrication process of various platforms that are used in the Examples below. Si wafers 17 are initially cleaned and then dipped in diluted acidic solution to remove any native oxide 104. The Si wafers 17 are then subjected to three different processes (210, 220 and 230) to generate the flat Si, IL-CE and GLAD-MACE platforms. Process 210 results in the production of a flat silicon substrate 17 with a silicon oxide surface 19.

Process 220 results in the IL-CE platform. Here, photoresist 21 is spin-coated onto a bare silicon 17 and cured. The exposed photoresist 21 is then removed and a metal catalyst 23 is thermally evaporated onto the photoresist 21. The substrate is then catalytically etched to remove portions of the substrate not covered by the photoresist to form nanostructures 30. The metal catalyst is then removed by standard etchant. The resultant substrate with the nanostructures 30 is then oxidized to form a layer of silicon oxide 19. Process 230 results in the GLAD-MACE platform. The substrate 17 is placed at an angle to the direction of the incoming metal catalyst 23 flux and rotated to cause the random deposition of the metal catalyst 23 particles on the surface of the substrate 17. The substrate 17 is then catalytically etched to form nanostructures 32. The metal catalyst 23 is then removed by standard etchant. The resultant substrate with the nanostructures 32 is then oxidized to form a layer of silicon oxide 19.

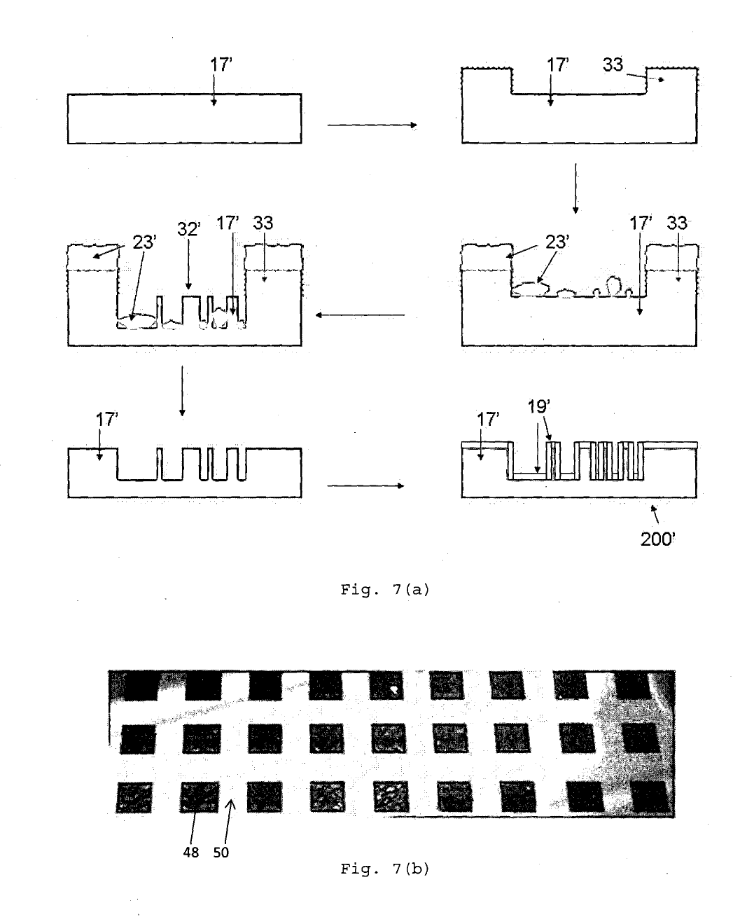

FIG. 7a depicts the fabrication of the GLAD-MACE microarray chip, namely ASANA 200′. Here, like reference numerals that are present in the above figures are repeated here but with the prime (′) symbol. A substrate 17′ that has been treated to remove any native oxide is placed into contact with a photoresist 33. The photoresist 33 is patterned by having openings of a desired dimension. The substrate 17′ is then subjected to a GLAD step for deposition of metal catalyst 23′ particles thereon. The substrate 17′ is then subjected to a catalytically etching step to cause areas of the substrate 17′ that are in contact with the metal catalyst 23′ to be etched away, forming nanostructures 32′ from regions that are not covered by the metal catalyst 23′. The photoresist 33 and metal catalyst 23′ are then removed and the resultant substrate is oxidized such that a layer of silicon oxide 19′ is formed on all of the exposed parts of the nanostructures and substrate.

FIG. 11 is a schematic diagram showing the use of an alternative ASANA platform based on optical activation. Here, a substrate 400 having a surface covered by the porous nanostructures is used. The substrate 400 is subjected to a first optical mask 52 which has exposed portions 53 that determines the detection regions. Light 54 is then used to link or graft the active agents 58 to the substrate 400. The same occurs when the substrate 400 is subjected to a second optical mask 56 which similarly, has exposed regions 55 that correspond to the detection regions that are to be activated by light 54 in order to link or graft a second set of active agents 60. In this manner, all of the active agents (58,60,62,64,66) on the microarray can be linked or grafted.

In use, when a sample containing desired target analytes (68a,68b,68c,68d,68e) are contacted with a solution containing the various intermediary linkers (70a,70b,70c,70d,70e) that are specific for the target analytes (68a,68b,68c,68d,68e), the intermediary linkers (70a,70b,70c,70d,70e) bind with the target analytes (68a,68b,68c,68d,68e) to form corresponding complexes (72a,72b,72c,72d,72e). The corresponding complexes (72a,72b,72c,72d,72e) then hybridize with the corresponding active agents (58,60,62,64,66) and the hybridization can be detected by a detector (not shown) using conventional detection methods. In this manner, it is possible to selectively activate detection regions while masking others that are not to be used for a certain sample.

### EXAMPLES

Non-limiting examples of the invention will be further described in greater detail by reference to specific Examples, which should not be construed as in any way limiting the scope of the invention.

In the following examples, hydrogen peroxide, HF, NH₄OH and HCl were obtained from Megachem Ltd (of Singapore); PDMS was produced from Sylgard 184 silicone elastomer kit from Dow Corning (of Michigan, United States of America); gold etchant was obtained from Sigma-Aldrich (of Missouri of the United States of America); DNA (sense and antisense) was obtained from 1^{st} Base (of Singapore), protein (antibody and antigen) was obtained from Thermo Scientific (of Massachusetts of the United States of America); EDC, HNSA, Cy3 and Cy5 were obtained from Pierce (under Thermo Scientific); and silicon wafer was obtained from Trading Resource.

### Example 1

### Basic Structure of ASANA in a Microfluidic Device

ASANA (200) is created on a specially designed nanostructured Si platform fabricated using Glancing Angle Deposition-Metal Assisted Catalytic Etching (GLAD-MACE) method and incorporated in a microfluidic device (300) as shown in FIG. 1. The microfluidic device (300) can be used for high-density capture and detection of target analytes (15) such as proteins or peptides as depicted in FIG. 1. Here, the ASANA is placed in a polydimethylsiloxane housing (9) with a channel (5) therethrough.

### Example 2

### Functionalization of ASANA

FIG. 2 shows the functionalization of the nanostructure clusters (2) present on an oxidized GLAD substrate (100) and the eventual detection of a target analyte (24). The oxidized GLAD substrate (100) or oxidized GLAD-MACE platform was first aminated with 2% 3-aminopropyltriethoxysilane. The high-density amine-modified surface (101) was then carboxylated (102). The carboxylated substrate (102) was then subjected to an incubation step with active agents (14) such as single stranded sense oligonucleotides that are coupled to a label such as Cy3 (16). The single stranded sense oligonucleotides with 5′-amino modifications are coupled to the carboxyl-terminated surface of the carboxylated substrate (102).

The substrate is then exposed to a solution containing a target analyte (24) such as a protein or peptide that is coupled to a second label molecule (22) such as Cy5. The target analyte (24) binds to an intermediary linker made up of an antibody (20) conjugated to a single stranded antisense oligonucleotide (18) when in solution. The complex made up of the target analyte (24) and intermediary linker then hybridizes with the single stranded sense oligonucleotide (14) in a hybridization step (12) due to complementary base pairing between the single stranded sense oligonucleotide (14) and the single stranded antisense oligonucleotide (18). The indirect binding of the target analyte (24) to the active agent (14) can be detected by the signal given off by the label (22). In this manner, the substrate can be used as a protein microarray.

By using the disclosed microarray, unfavorable interfacial interactions between the intermediary linker and substrate surface and diffusion-limited capture of the target can be avoided. In addition, the microarray can be tailored to test for a wide range of target analytes.

### Example 3

### Fabrication of Flat Si, IL-Ce and GLAD-MACE Platforms

Three types of substrates were fabricated according to the processes depicted in FIG. 3.

Firstly, N-type Si wafers (17) having a resistivity of 10 Ωcm were used. The wafers were first subjected to a 1 minute dip in 10% HF solution to remove any native oxide presents for cleaning.

For process 210, a thin oxide layer 19 was thermally grown on the Si wafer (17). This oxidized flat Si surface acts as a control for the different nanostructured surfaces.

For the IL-CE process (22), the Si wafer (17) was coated with a layer of photoresist (21) such as Ultra-i 123 photoresist until a thickness of approximately 400 nm. The coated Si wafer was then cured at 90° C. for 90 seconds. The photoresist was exposed using a Lloyd's-mirror-type IL set-up with a HeCd laser source with two perpendicular exposures of approximately 40 seconds to 1 minute. The exposed photoresist was removed using the Microposit MF CD-26 developer leaving behind circular-shaped photoresist dots on the Si wafer surface. Metal catalyst (23) such as Au was thermally evaporated on the substrate to a thickness of about 25 nm at a pressure of 10⁻⁶ Torr. The samples were then etched in a solution of H₂O, HF and H₂O_{2 at room temperature, with the concentrations of HF and H}₂O₂ fixed at 4.6 and 0.44 M, respectively, resulting in ordered Si nanopillars on the Si surface. The Au was removed using a standard Au etchant, followed by oxidation in O₂ at 900° C. for 35 min.

For the GLAD-MACE process (230), the Si wafer (17) was placed in an electron-beam evaporator. The chamber of the electron-beam evaporator was pumped down to a pressure of 10⁻⁶ Torr before commencing the GLAD process. The substrate normal was placed at an angle of 87° to the direction of the incoming Au flux and the substrate was rotated at a rate of 0.2 rpm to allow the metal catalyst (23) particles such as Au particles to be deposited on the surface of the Si wafer (17). The samples were then etched in a solution of H₂O, HF and H₂O₂ at room temperature with the concentrations of HF and H₂O₂ fixed at 4.6 and 0.44 M, respectively. The Au on the Si surface was then removed using a standard Au etchant and followed by oxidation in O₂ at 900° C. for 35 min. The above steps were carried out in a class 10 000 cleanroom.

The GLAD-MACE process produces randomly distributed and thinner Si nanowires (about 10-100 nm in diameter) as compared to the highly ordered and thicker (about 200-400 nm in diameter) nanopillars synthesized by the IL-CE method. Therefore, the substrate obtained from the GLAD-MACE method had a much higher nanowire density per unit area as compared to the substrate produced by the IL-MACE method.

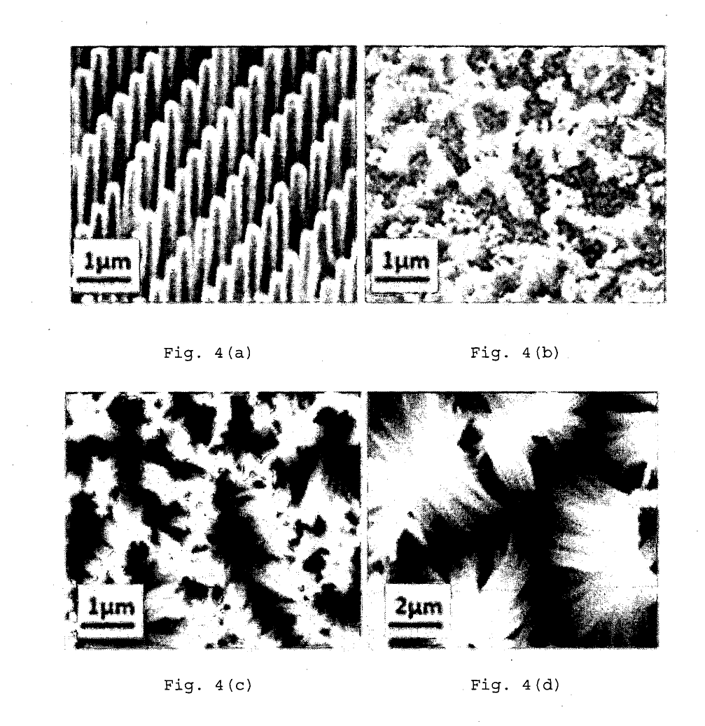

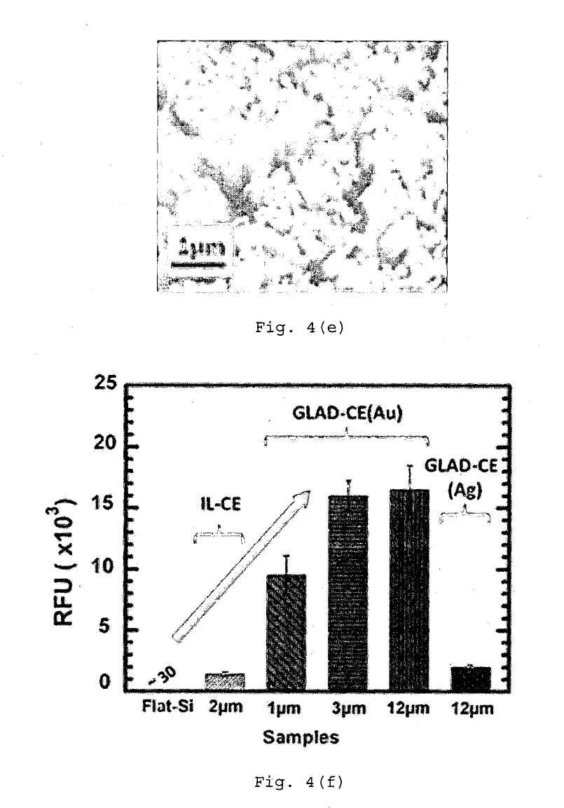

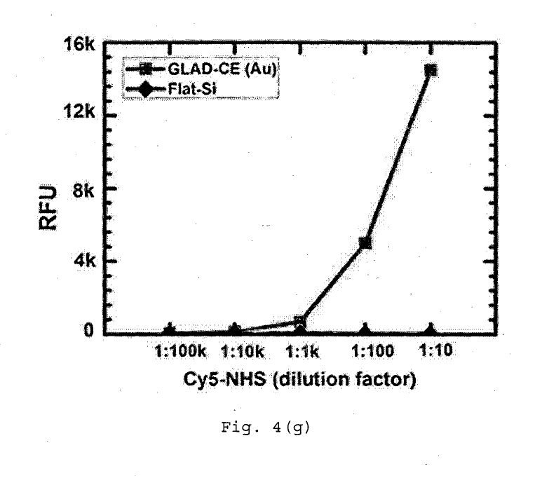

FIG. 4 shows SEM images of the various platforms used to test the performance of ASANA. FIG. 4(a) is an SEM image of Si nanopillars fabricated via the IL-CE process (220), while FIG. 4(b) to FIG. 4(d) are SEM images of Si nanostructures fabricated via the GLAD-MACE process (230) using Au as a catalyst. As can be seen in these figures, the nanostructures form clusters on the substrate. FIG. 4(e) is a SEM image of Si nanostructures fabricated via the GLAD-MACE process (230) using Ag as a catalyst. There is also a degree of clustering of the nanostructures of FIG. 4(e), albeit at a lower extent as compared to the nanostructures depicted in FIG. 4(d).

The Si nanostructures in the form of nanopillars fabricated via the IL-CE process (220) typically have a diameter of approximately 400 nm and heights of up to 2 μm, whereas the GLAD-MACE process (230) results in a surface made up of nanostructures in the form of nanowires with diameters of approximately 10 to 100 nm and heights of from 2 μm to 12 μm.

### Example 4

### Technical Validation via Coupling Efficiency of GLAD-CE Platform with Cy5 (Performance Test 1)

### Comparison Between the Flat Si, IL-Ce and GLAD-MACE Platforms

Surface density of reactive groups is critical for development of high-density microarray for detection of DNA and protein molecules. Evaluation of the density of carboxylic acid groups (after carboxylation) on both flat and nanostructured surfaces by direct coupling to Cy5-NHS ester is shown in FIG. 4(f). Flat-Si surface was found to display minimal coupling to Cy5. The nanopillars of 2 μm height (from the IL-CE process) exhibited 40 times improvement on Cy5 coupling. A far significant increase in Cy5 coupling (approximately 300-600 fold higher than that of the flat surface) was observed on all the nanostructured surfaces fabricated via GLAD-MACE, with varied heights from 2 μm to 12 μm. Furthermore, coupling of amine groups on GLAD-MACE surface to serial 10-fold dilutions Cy5-NHS showed far greater signal intensity over large dynamic range when compared to that of a flat oxidized Si surface (FIG. 4g).

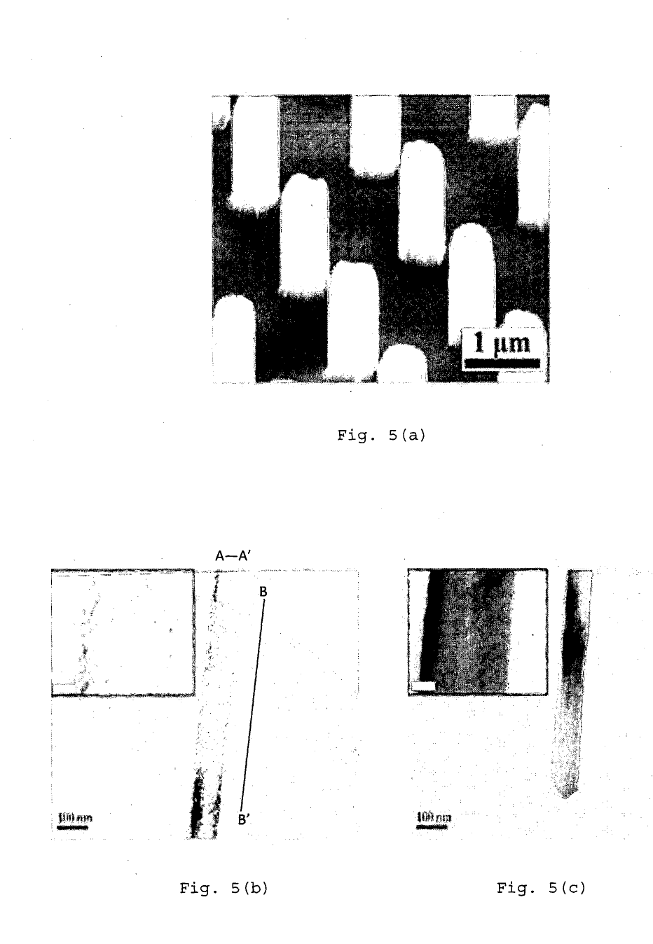

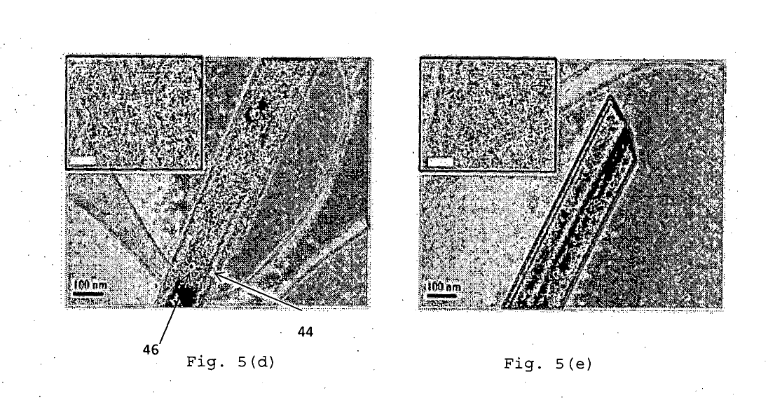

The massive increase in Cy5 coupling on the GLAD-MACE surface can be attributed to an increased surface area due to surface roughness of nanowires. FIG. 5a shows the SEM image of nanopillars prepared by the IL-CE method; the figure shows an irregular tip and textured cylindrical surface. Thus, a 40-fold increase in the coupling efficiency of Cy5 of the IL-CE nanopillars can be traced to the surface roughness.

An obvious difference between the IL-CE nanopillars and the GLAD-MACE nanowires is that the IL-CE nanopilllars stand upright from the Si surface while the GLAD-MACE nanowires tend to coalesce. FIG. 5(b) and FIG. 5(c) show the TEM images of the top and bottom sections of a nanowire obtained from the GLAD-MACE method using Au catalysts. The HRTEM images of the respective section of the nanowire (see insets) show that the top part of the nanowire is more porous than that of the bottom part. As can be seen from FIG. 5(b), the network of pores extends throughout the thickness of the nanostructure (as depicted by the line A-A′). In addition, the network of pores also extends throughout the height of the top part of the nanostructure (as depicted by the line B-B′). The porous top part of the nanowire tends to stick together by the capillary force and short-ranged van der Waals force when the sample were left to dry after etching. FIG. 5(b) and FIG. 5(c) also show that as the porosity of the GLAD-MACE nanowires is much higher than that of the IL-CE nanowires (FIG. 5(a)), the coupling efficiency of the GLAD-MACE platform to Cy5 is greatly enhanced as compared to the IL-CE platform.

Comparison of GLAD-MACE Platforms Obtained from Au and Ag Catalysts

The Cy5 coupling efficiency of GLAD-MACE platforms obtained from the GLAD-MACE process with Au and Ag catalysts was compared. FIG. 5(d) and FIG. 5(e) are TEM images of the top and bottom, respectively, of a nanowire obtained by using Ag catalysts with exactly the same etching conditions as that used for Au catalysts. The nanowire obtained from the Ag catalysts is less porous as compared to the nanowire obtained from Au catalysts (see FIG. 5(b) and FIG. 5(c)). FIG. 5(d) and FIG. 5(e) show that the top and bottom parts of the Ag-etched nanowires were less porous as compared to the corresponding top (FIG. 5(b) and bottom parts (FIG. 5(c)) of the Au etched nanowires.

In comparing the nanowire from FIG. 5(b) and FIG. 5(d), it can be seen that the top portion of the Au-etched nanowire is porous such that the pores extend throughout the thickness (as defined by the width dimension) of the nanowire. However, in FIG. 5(d), the Ag-etched nanowire is only porous in the outer portion 44 of the nanowire and that there is a silicon core 46 in the nanowire that is not porous. Hence, the Ag-etched nanowire does not have pores that extend throughout the thickness (as defined by the width dimension) of the nanowire. It is to be noted that there is no such core-shell configuration in the Au-etched nanowire. Due to the higher electronegativity of Au as compared to Ag, the Au catalysts trap more holes during the etching step as compared to silver, leading to more pores being formed in the Au-etched nanowire. Due to the lower porosity of the Ag-etched nanowire, the Cy5 coupling efficiency is lower as compared to that obtained from the Au-etched nanowire.

Due to the lower porosity of the Ag-etched nanowire, the Ag-etched nanowires are more rigid and tend to cluster to a lesser extent after the GLAD-MACE process was completed. The coupling efficiency data of Cy5 on the substrate obtained from the Ag catalysts can be seen in FIG. 4(f). The improvement of Cy5 coupling efficiency of Ag-etched nanowires is 60-fold. As the Ag-etched nanowires (FIG. 5(d) and FIG. 5(e)) are less porous than the Au-etched nanowires (FIG. 5(b) and FIG. 5(c)), it is clear that the porosity of nanowires plays a crucial role in determining the coupling efficiency of Cy5.

Comparison of GLAD-MACE Platform with Roughen Si Surface

The performance of GLAD-MACE platform was compared with a roughened Si surface. Here, a 2 to 3 nm Au film was thermally evaporated on the Si substrate. The wafer was then subjected to a MACE process in H₂O₂ and HF to produce a roughened Si surface.

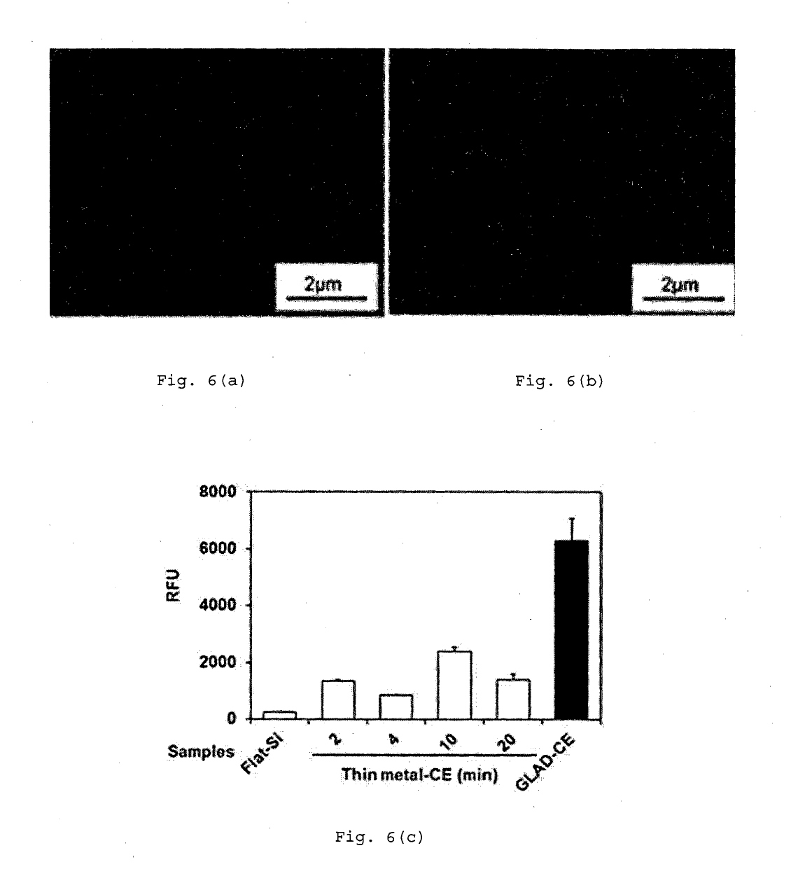

By varying the etching durations from 2 to 20 minutes, roughened surfaces with heights from 0.5 μm to 5 μm can be fabricated (see FIG. 6(a) and FIG. 6(b)). A short etching duration resulted in a roughened Si substrate while a longer etching duration resulted in nanostructured surfaces made up of nanowires and nanowalls. FIG. 6(c) shows that although the roughened surfaces show some improvement on Cy5 coupling, none of them could achieve the high signal intensity demonstrated by the GLAD-MACE surfaces. For a short etching duration, the simply roughened surface lacked the density and aspect ratio of the GLAD-MACE platform. Although nanowire arrays were achievable with a longer etching duration, the sinking of the discontinuous thin film produced a lower density of nanowires compared to that obtained from GLAD-MACE process. These results highlight the uniqueness and the superiority of the GLAD-MACE platform for Cy5 coupling compared to a roughened Si surface.

### Example 5

### Technical Validation via Optimization of Etching Conditions for Platform Fabrication (Performance Test 2)

The effect of catalytic etching conditions on the morphology of the nanowires prepared by the GLAD-MACE method with Au catalysts was investigated. The morphology and porosity of the nanostructures are closely related to the concentration of chemical agent and etching temperature.

An increase of [H₂O₂] from 0.97M to 4.4M with a fixed [HF] resulted in longer nanowires, and the nanowires clustered earlier during the etching process and form larger size of clusters. “Ribbon-like” nanostructures were obtained under the condition of very high [H₂O₂]. The change in morphology of the nanowire surfaces due to varying [H₂O₂] can be attributed to an increase of porosity which has been confirmed by our Raman and TEM results. As [HF] increases to 10M with fixed [H₂O₂], longer and straighter wires were obtained. Increase of etching temperature to 50° C. led to more sparse and translucent “coral-like” nanostructures. The nanowires etched at elevated temperature were shorter and more porous than those etched at room temperature because a higher H₂O₂ decomposition at higher temperature made the Si etching more efficient.

### Example 6

### Fabrication of ASANA Microarray

FIG. 7(a) schematically illustrates the fabrication of the GLAD-MACE microarray chip, namely, ASANA. First, square openings of desired dimension were patterned on photoresist (33) on Si (17′) using conventional photolithography. Next, a GLAD process was performed to deposit the Au particles (23′). The substrate was then subjected to MACE in order to form the nanostructures (32′). The Au particles (23′) were removed and the resultant Si wafer was oxidized to form an oxide layer (19′) on the surface. The final wafer was then cut to a dimension to fit into an array scanner. FIG. 7(b) illustrates the finished ASANA microarray. As can be seen in FIG. 7(b), the detection regions 48 are spatially distinct and separated from each other by substrate banks 50.

The fabrication of the ASANA microarrays enforces (i) the compatibility of the GLAD-MACE process with conventional microelectronics processes such as lithography; (ii) the ability to spatially determine the size and position of the desired testing area, i.e. scalability; which allows the possible fabrication of thousands of testing sites per chip; and (iii) lower cost required to fabricate such a device since complex lithography and etching techniques such as e-beam lithography and reactive ion etching are not used.

In addition, the ASANA design will allow, among other things, (i) incorporation of flowing the target analyte solution along the detection platform to surpass and overcome diffusion-limited capture and detection to thereby enhance efficiency and speed, (ii) precise control of amount of loading, dynamic alteration of formulation chemistry, control of micromixing, etc, as needed, and (iii) real-time changes, as needed, in these operating parameters.

### Example 7

### Working Example of ASANA—DNA Coupling and Detection

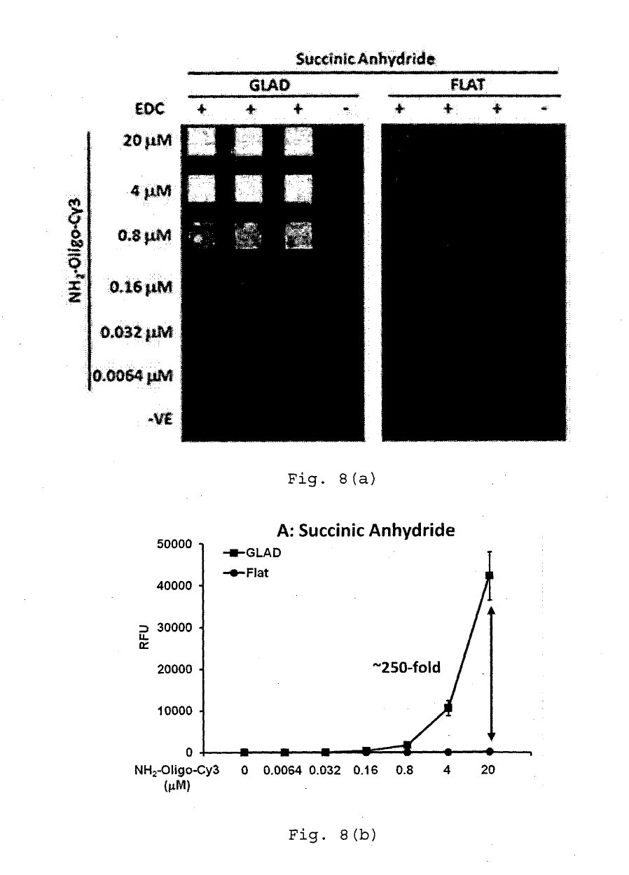

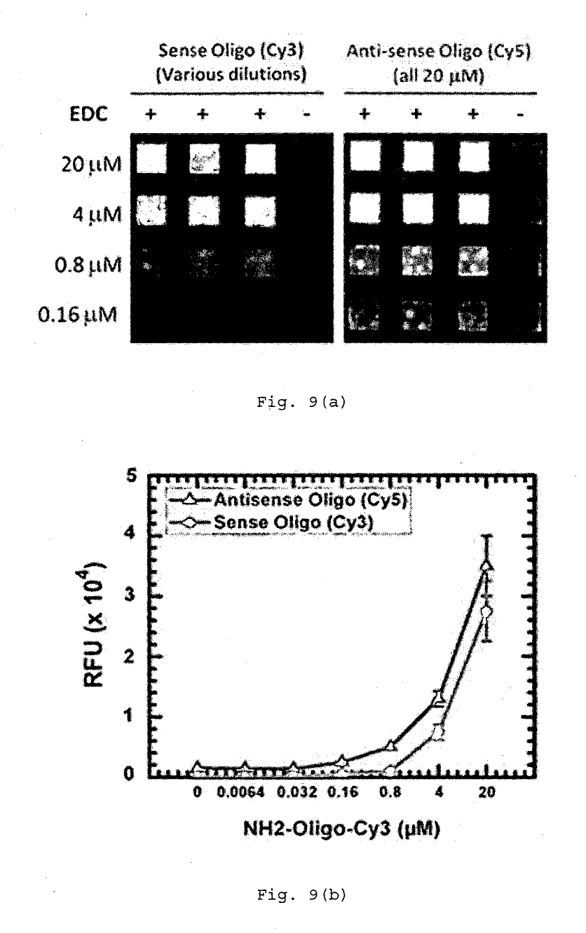

In both DNA and protein microarrays, single-strand DNA oligos (ssDNA) were immobilized onto the base platform, which allows sequence specific capturing of either the target DNA or complementary ssDNA-conjugated probes. The loading capacities of single-strand DNA (ssDNA) on GLAD-MACE and flat-Si chip were compared as disclosed in FIG. 8. Both surfaces are aminosilanized and further functionalized with a linear linker succinamic acid to enable loading of an amine terminated, Cy3 coupled ssDNA (NH₂-Oligo-Cy3). GLAD-MACE surface showed dose-dependent coupling of the Cy3-oligo (6.4 nM to 20 μM) (FIG. 8). The control reaction without cross-linker EDC (any heterobifunctional, water-soluble, zero-length carbodiimide crosslinker that was used to couple carboxyl groups to primary amines) confirmed that the coupling was not due to non-specific adsorption of the oligonucleotides onto the GLAD-MACE surface. Flat Si surface, in contrast, had significantly lower coupling efficiency under all conditions. The GLAD-MACE surface showed approximately 250 fold increase in signal intensity compared to that of the flat Si surface.

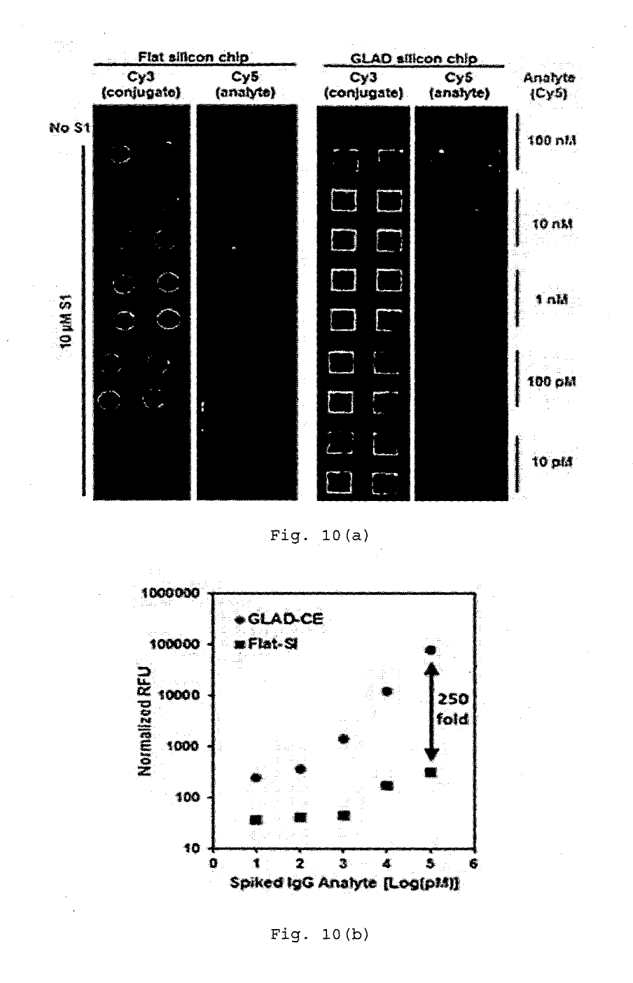

The efficiency of the GLAD-MACE chips for DNA immobilization for target detection has also been determined. For this, the chips were functionalized with a Cy3 coupled ssDNA (NH2-Oligo-Cy3). Next the chips were loaded with a complimentary, Cy5 coupled ssDNA (anti-sense oligonucleotide). As shown in FIG. 9, the antisense oligo showed a corresponding trend with the complimentary sense oligonucleotide. A high coupling efficiency equivalent to the Cy3 sense oligonucleotide was preserved. The data presented shows that GLAD-MACE surface is a more superior base platform than the flat Si surface.

### Example 8

### ASANA Based Protein Chip

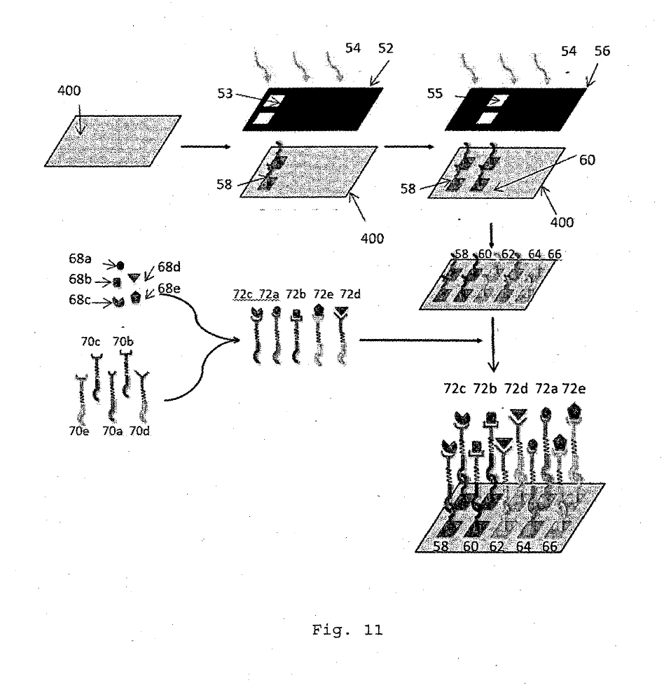

The GLAD-MACE platform (the ASANA chip) was used in detecting protein analytes from complex biological fluids. Human serum was spiked with different concentration of the analyte of interest, a model analyte rabbit IgG (Cy5 labeled rabbit IgG, 10 pM to 100 nM). The performance of ASANA chip was tested by homogeneous phase capturing of the Cy5-labeled rabbit IgG using ssDNA conjugated goat anti-rabbit antibody (ASR) followed by self-assembly of the analyte-ASR complex on complementary ssDNA functionalized GLAD-MACE and flat substrates. For the results depicted in FIG. 10, the analytes were captured by goat anti-rabbit antibody conjugated with Cy3 labeled anti-sense oligonucleotide 1 (ASR, 0.5 μM). The resulting analyte-ASR complex was allowed to hybridize to sense oligonucleotide 1 functionalized flat and ASANA arrays.

A dose-dependent detection of the analyte was observed on both substrates (FIG. 10(a)). The control reaction on substrate not functionalized with ssDNA confirmed that the hybridization was not due to non-specific adsorption of the analyte or ASR onto the GLAD-MACE substrate. Normalized RFU (analyte/ASR) showed that GLAD-MACE substrate captured significantly more analyte than flat substrate (up to 250 fold, FIG. 10(b)). These results indicate that the ASANA chip offers higher loading capacity and an improved signal-to-noise ratio, and can be adapted for the detection and quantification of various types of biomolecules in complex biological samples.

### Example 9

### Effect of Drying Media

In order to assess the effect of drying media on the clumping effect and hence surface area of the nanostructure clusters, a set of Au-etched GLAD-MACE nanostructures were dried in de-ionized water, methanol and de-ionized water with N₂ flow (that is, dried with a nitrogen gun). The nanostructures were dried in the respective media after the etching step. The nanostructures were dried in de-ionized water and methanol in ambient environment for 24 hours and in de-ionized water with N₂ flow for 1 minute. In order to estimate the “exposed” surfaces of the nanostructure clusters for Cy5-NHS coupling, a software package (ImageJ, developed by the National Institutes of Health of the United States Department of Health and Human Services) was used. The exposed surface was determined by the perimeter per unit area of the clustered nanostructures. It can be seen from Table 1 that the magnitudes of the perimeter per unit area obtained from the samples dried with methanol and N₂ flow are higher than that from the sample dried in de-ionized water. The Cy5-NHS coupling efficiencies of the various nanostructure clusters are also shown in Table 1. As all of the nanostructures have the same porosity, the samples with a larger exposed surface (that is, a higher value of perimeter per unit area) will have a higher Cy5-NHS coupling efficiency.

Another sample that was made using Ag as the etching catalyst and dried in de-ionized water with N₂ flow was also investigated. The perimeter per unit area and Cy5-NHS coupling efficiency of this sample are also shown in Table 1. It can be noted that although the magnitude of the perimeter per unit area of the less porous nanostructures obtained from the Ag catalysts was the highest, the Cy5-NHS coupling efficiency of this sample was much lower than those obtained with the Au catalysts. Hence, this indicates that other than the surface area, porosity also plays an important role in enhancing the coupling efficiency of Cy5-NHS on the GLAD-MACE nanostructure clusters. Hence, the immobilization efficiency of the active agents on the nanostructure clusters can also be affected by the surface area and/or porosity of the nanostructure clusters.

TABLE 1

| Sample | | Perimeter per unit | Cy5-NHS coupling |
|---|---|---|---|
| Catalyst | Drying process | area/μm⁻¹ | efficiency |
| Au | De-ionized water | 1.82 | 6.67 × 10³ |
| Au | De-ionized water | 2.09 | 1.36 × 10⁴ |
| | with N₂ flow | | |
| Au | Methanol | 1.92 | 9.73 × 10³ |
| Ag | De-ionized water | 7.66 | 2 × 10³ |
| | with N₂ flow | | |

### Applications

The disclosed method can be used to form microarrays on a large-area and highly scalable platform. The disclosed method can be combined with conventional photolithography to fabricate the microarray. The disclosed method may not require the use of complex lithography or etching techniques such as electron-beam lithography or reactive ion etching to form the nanostructures, leading to savings in cost.

The microarray can be used for clinical and research in vitro assays. Advantageously, the disclosed microarray may mitigate interfacial limitations due to heterogeneous phase interactions of analyte-surface interactions. The microarray may be highly-specific and may have a high signal-to-noise ratio so that sensitive and reliable detection of extremely low levels of target(s) may be possible. The microarray may be used to detect and quantitate various types of targets such as target proteins, peptides, nucleic acids and small molecules in pico-molar range without amplification. The microarray can be used as a DNA-directed homogeneous-phase analyte-capture platform for detection and quantification of a number of biological targets.

The microarray can be used to house unlimited active agents that allow molecular recognition of a specific molecule or reaction of interest with high throughput, high specificity and enhanced signal-to-noise ratio. The microarray can be used to screen for a large number of targets, leading to high throughput.

Due to the increased surface area contributed by the nanostructure clusters, the active agents can be accessible to the targets without suffering from the drawbacks of the prior art such as electrostatic hindrance or unwanted interactions between the active agents.

It will be apparent that various other modifications and adaptations of the invention will be apparent to the person skilled in the art after reading the foregoing disclosure without departing from the spirit and scope of the invention and it is intended that all such modifications and adaptations come within the scope of the appended claims.

1. A microarray comprising a plurality of active agents immobilized onto an array of porous nanostructures, wherein each nanostructure has a network of pores that extend throughout at least one dimension of said nanostructure.

2. The microarray according to claim 1, wherein the size of each pore is in the range of 0.1 nm to 10 nm.

3. The microarray according to claim 2, wherein a plurality of porous nanostructures cluster together to form a nanostructure cluster, wherein in each nanostructure cluster, the distal ends of said porous nanostructures are spaced closer to each other relative to the respective proximal ends of adjacent nanostructures.

4. The microarray according to claim 1, wherein the density of said active agents on said substrate is in the range of 1×10³ mm⁻² to 1×10¹⁸ mm⁻².

5. The microarray according to claim 1, wherein the immobilization efficiency of said active agents to said porous nanostructures is increased by at least 60 fold as compared to the immobilization efficiency of identical active agents to a substrate not having the nanostructures thereon.

6. The microarray according to claim 1, wherein said active agents are immobilized to said nanostructures via a linker molecule.

7. The microarray according to claim 1, comprising a plurality of detection regions on said substrate, wherein each detection region comprises active agents immobilized to said array of porous nanostructures, and wherein each detection region comprises a specific type of active agents that are the same as or different between the detection regions.

8. A method of forming a microarray comprising the step of immobilizing active agents to an array of porous nanostructures, wherein each nanostructure has a network of pores that extend throughout at least one dimension of said nanostructure.

9. The method according to claim 8, comprising the step of, before said immobilizing step, providing said array of porous nanostructures on a substrate.

10. (canceled)

11. The method according to claim 9, wherein the providing step comprises the step of selectively etching said substrate.

12. (canceled)

13. The method according to claim 11, comprising the step of, before the etching step, contacting part of the area of said substrate with a plurality of catalyst particles that promote the rate of etching when said substrate is exposed to an etchant while leaving the remainder of the area of said substrate not exposed to said catalyst particles.

14. The method according to claim 11, comprising the step of, after said etching step, drying said porous nanostructures to thereby cause said porous nanostructures to cluster together to form a nanostructure cluster, wherein in each nanostructure cluster, the distal ends of said porous nanostructures are spaced closer to each other relative to the respective proximal ends of adjacent nanostructures.

15. (canceled)

16. (canceled)

17. (canceled)

18. (canceled)

19. (canceled)

20. The method according to claim 8, comprising the step of selecting a polynucleotide as said active agent.

21. (canceled)

22. (canceled)

23. The method according to claim 8, further comprising the step of forming a plurality of detection regions on said substrate, each detection region comprising active agents immobilized to said array of porous nanostructures, wherein each detection region comprises a specific type of active agents that are the same as or different between the detection regions.

24. The method according to claim 23, wherein said forming step comprises the step of subjecting the substrate to a lithography technique to form patterns on the substrate that determine the positions of the detection regions.

25. A system for detecting the presence or absence of a target in a sample, comprising:
a microarray comprising a plurality of active agents immobilized onto an array of porous nanostructures, wherein each nanostructure has a network of pores that extend throughout at least one dimension of said nanostructure, and wherein said active agents have an affinity for said target and are coupled to a label to produce a signal when bound to said target; and
a detector for detecting the signal produced by said label to determine the presence or absence of said target in said sample.

26. (canceled)

27. The system according to claim 25, wherein said active agent is a polynucleotide.

28. (canceled)

29. (canceled)

30. (canceled)

31. (canceled)

32. The system according to claim 25, wherein said target is an analyte that has an affinity for said polypeptide and is coupled to a label.

33. A microfluidic device for detecting the presence or absence of a target in a sample, comprising:
a microarray comprising a plurality of active agents immobilized onto an array of porous nanostructures, wherein each nanostructure has a network of pores that extend throughout at least one dimension of said nanostructure, and wherein said active agents have an affinity for said target and are coupled to a label to produce a signal when bound to said target;
a channel for directing the sample flow towards said microarray; and
a detector for detecting the signal produced by said label to determine the presence or absence of said target in said sample.

34. A method of making a microarray comprising the steps of:
contacting part of the area of an etchable substrate with catalyst particles that promote the rate of etching when said substrate is exposed to an etchant while leaving the remainder of the area of the substrate not exposed to the catalyst particles;
etching the substrate in the presence of an etchant to form porous nanostructures thereon from areas of the substrate that are not exposed to the catalyst particles, wherein each nanostructure has a network of pores that extends throughout at least one dimension of said nanostructure;
removing the etchant from the substrate to form an array of porous nanostructures on the substrate; and immobilizing active agents to the array of nanostructure clusters.

35. (canceled)
